(19) United States
(12) Patent Application Publication
(10) Pub. No.: US 2024/0024693 A1
(43) Pub. Date: Jan. 25, 2024

(54) SYSTEM FOR APPLYING MAGNETIC FIELD TO ANESTHETIZE A PATIENT

(71) Applicant: Henry Gonzalez, Upland, CA (US)

(72) Inventor: Henry Gonzalez, Upland, CA (US)

(21) Appl. No.: 17/870,762

(22) Filed: Jul. 21, 2022

(51) Int. Cl. A61N 2/00; A61N 2/12

(52) U.S. Cl. CPC A61N 2/008; A61N 2/12; A61N 2/006

(57) ABSTRACT

A system for applying magnetic field to anesthetize a patient is disclosed. The system comprises a first plurality of magnets positioned below a surgical bed to apply the magnetic field when the patient lies on the surgical bed; a second plurality of magnets coupled to one or more reciprocating units and provided operatively below the surgical bed, and one or more ultrasound probes positioned between the first plurality of magnets to measure the spinal cord's depth. The reciprocating units are configured to facilitate a reciprocal movement the second plurality of magnets, thereby moving the second plurality of magnets towards and away from the patient's body for facilitating variation of the magnetic field provided to the patient. The variation of the magnetic field induces an electric flux within the spinal cord, thereby disrupting neurons in the spinal cord and preventing the patient from feeling any pain during a surgical procedure.

### TECHNICAL FIELD

The present invention generally relates to magnetic therapeutic systems. More specifically, the present invention relates to a system for applying a magnetic field to anesthetize a patient, thereby preventing the patient from feeling any pain/pressure during a surgical procedure.

### BACKGROUND

Medical industry today includes various devices and systems for treating patients with different conditions. Among such devices and systems, magnets are used in many therapeutic applications. Magnets are also essential in operation of many medical machines such as MRI machines and in other medical technologies. Another application of magnets is that they can be used mainly in improving nerve functions and alleviation of pain in patients.

Using magnets for anesthetizing patients may facilitate provision of a non-invasive anesthesia means. However, none of the existing solutions disclose a system for anesthetizing a patient using a magnetic field. In addition, none of the existing solutions disclose a system to anesthetize a patient via a changing magnetic field to prevent the patient from experiencing any pain during a surgical procedure.

As such, there is a need for a system for anesthetizing a patient using a magnetic field. Also, there is a need for a system for anesthetizing a patient via a changing the magnetic field to prevent the patient from experiencing any pain during a surgical procedure.

### SUMMARY

The present invention generally discloses magnetic therapeutic systems. More specifically, the present invention relates to a system for applying a magnetic field to anesthetize a patient, thereby preventing the patient from feeling any pain during the surgical procedure.

According to the present invention, the system is configured to apply the magnetic field when the patient lies on the surgical bed. In one embodiment, the system comprises a first plurality of magnets positioned below a surgical bed/table of the patient to apply the magnetic field when the patient lies on the surgical bed. In one embodiment, the first plurality of magnets includes one or more permanent magnets and one or more electromagnets.

In one embodiment, the permanent magnets may be connected to pistons or other comparable devices to facilitate an off-axis rotational movement or wobbling movement (back and forth movement relative to the long axis of the surgical bed) thereof. In one embodiment, the permanent magnets effectuate the varying magnetic field into the patient's body including the spinal cord and ventral and dorsal roots of the spinal nerves.

In one embodiment, at least one electromagnet is placed at a first end of the surgical bed, and at least one electromagnet is placed at a second end of the surgical bed. In one embodiment, both the electromagnets include various on and off frequencies, which imparts a pulse wave pattern of magnetic field at both a cervical and a lower thoracic or upper lumbar region of the patient present on the surgical bed.

In one embodiment, the system further comprises a second plurality of magnets positioned on either side of or between the plurality of magnets. In one embodiment, the second plurality of magnets includes a plurality of interchangeable pyramidal magnets. In one embodiment, the second plurality of magnets are configured to move towards and away from the patient.

In one embodiment, the system further comprises one or more ultrasound probes that are positioned between the first plurality of magnets to measure the depth of a thoracic spinal cord of the patient. In one embodiment, the second plurality of magnets are coupled to one or more reciprocating units and provided operatively below the surgical bed. In one embodiment, the reciprocating units are configured for facilitating the reciprocal movement of the second plurality of magnets towards and away from the patient's body. In one embodiment, the reciprocal movement of the second plurality of magnets direct a magnetic field of the first plurality of magnets substantially perpendicular to the surface of the surgical bed for entering into the patient's body present in the surface of the surgical bed. In one embodiment, the second plurality of magnets are moved towards and away from the patient's body for facilitating the variation of the magnetic field provided to the patient. The change in the magnetic field induces an electric flux within the spinal cord and/or spinal nerve roots, thereby disrupting neurons in the spinal cord and preventing the patient from feeling any pain during a surgical procedure.

In one embodiment, the permanent magnets are powerful rectangular elongated magnets and also of Halbach design. The magnetic field is emanated from both types of magnets in a predictable and focused manner. In one embodiment, the magnetic field emanating from the magnets is directed toward the central spinal cord tissue and also along the ventral and dorsal root nerves. In one embodiment, the magnetic field emanating outwards has the ability to create an electric field flux within a circuit found within the spinal cord, and/or the spinal cord and spinal nerve roots. In one embodiment, the magnetic field is directed to a desired range of spinal cord including the lower cervical region through the upper thoracic region, or upper thoracic region through the upper lumbar region, etc.

In one embodiment, the system further comprises a first control panel configured to control one or more parameters of the first plurality of magnets and the second plurality of magnets. In one embodiment, the parameters include a speed at which the magnets reciprocate near the surface of the surgical bed. In one embodiment, the system further comprises a second control panel configured to collect one or more parameters of the patient. In one embodiment, the parameters may be the patient's BMI, height, weight, and girth (side to side width).

After the patient's parameters are entered into the second control panel, the ultrasound probe is moved into position. In one embodiment, the ultrasound probe is moved in a position configured to transmit a high-frequency sound wave around the patient's body for determining the depth of the patient's spinal cord. In one embodiment, the determined depth of the patient's spinal cord is compared to the pre-programmed indices that are provided in a memory of the second control panel. In one embodiment, the system further comprises a cooling mechanism. In one embodiment, the cooling mechanism is utilized for cooling the first and second plurality of magnets in order to amplify the power of the permanent magnets. In one embodiment, the cooling mechanism is configured to reduce the heat generated due to the action of repeated and rapid up and down movement or reciprocating movement of the second plurality of magnets. Further, the cooling mechanism is used to cool the first and second plurality of magnets to keep them functioning properly.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIGS. 1-2 show a system for applying a magnetic field to anesthetize a patient in an embodiment of the present invention.

FIG. 3 shows an arrangement of one or more components of the system in one embodiment of the present invention.

FIGS. 4-5 show an alignment of one or more magnets in one embodiment of the present invention.

FIG. 6 shows the rotation of the magnets in one embodiment of the present invention.

FIGS. 7A-7B show the angle of rotation of the magnets in one embodiment of the present invention.

FIG. 8 shows magnets in relation to a cross section of the spinal cord are shown to emphasize the direction of the magnetic flux in relation to the spinal cord.

FIG. 9 shows one or more parameters of the patient in one embodiment of the present invention.

FIG. 10 shows a direction of the magnetic field/flux towards the surgical bed in one embodiment of the present invention.

FIG. 11 shows a control panel of the system in one embodiment of the present invention.

FIG. 12 shows an ultrasound image of the patient's body in one embodiment of the present invention.

FIGS. 13-14 show different types of surgical bed in one embodiment of the present invention.

### DETAILED DESCRIPTION OF EXAMPLE EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

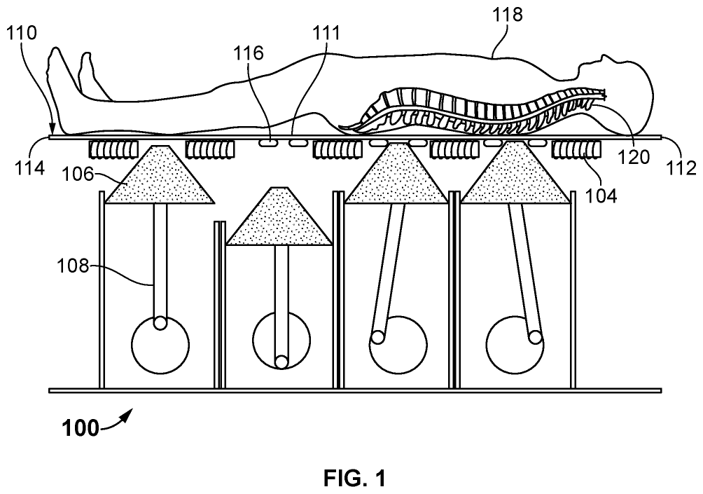

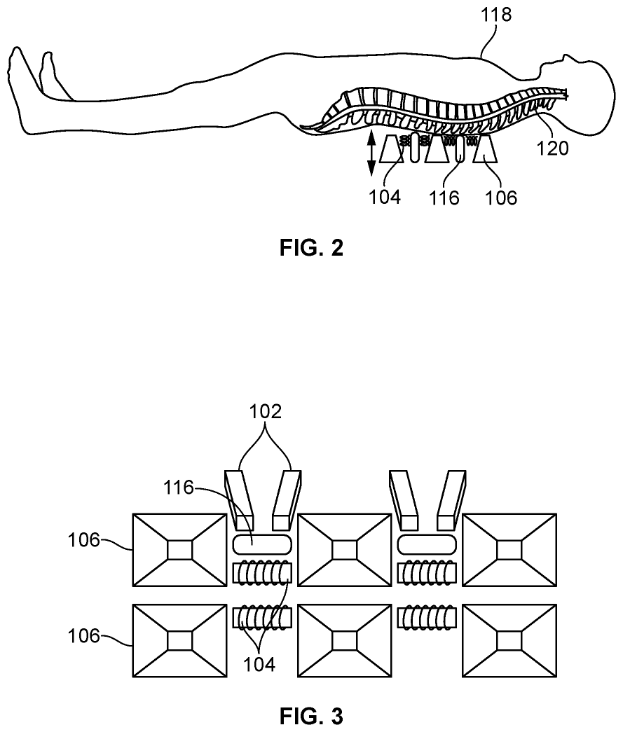

Referring to FIGS. 1-3, a system 100 and an arrangement of one or more components for applying a magnetic field/flux 128 (as shown in FIG. 5) to anesthetize a patient 118 is illustrated. In one embodiment, the system 100 comprises a first plurality of magnets positioned below a surgical bed/table 110 of the patient 118 to apply the magnetic field when the patient 118 is present on the surgical bed 110. In one embodiment, the first plurality of magnets includes one or more permanent magnets 102 (as shown in FIG. 3) and one or more electromagnets 104.

In one embodiment, at least one electromagnet 104 is placed at a first end 112 of the surgical bed 110, and at least one electromagnet 104 is placed at a second end 114 of the surgical bed 110. In one embodiment, both the electromagnets 104 have various on and off frequencies, which impart a pulse wave pattern of magnetic field 128 at both a cervical and a lower thoracic or upper lumbar region of the patient 118 present on the surgical bed 110. The magnetic flux pattern provided by the at least one electromagnet 104 is different from the waveform pattern of the magnetic flux imposed upon the tissue via the permanent magnets 102. The interaction of the magnetic fields generated by the permanent magnet 102 and the electromagnet 104 facilitates the variation in the magnetic field provided to the patient 118 in a manner similar to the variation in a magnetic field provided by a permanent magnet to an object while the permanent magnet is in oscillation (moves towards and away) with respect to the object. In one embodiment, an on/off of frequency of the electricity to the electromagnets 104 facilitates provision of the pulse wave pattern of the magnetic field. In one embodiment, the on/off frequency of the electromagnets 104 may be “ramped up and down” for facilitating the variation of magnetic field provided to the patient 118.

In one embodiment, the system 100 further comprises a second plurality of magnets 106 positioned on either side of or between the first plurality of magnets (102 and 104). In one embodiment, the second plurality of magnets 106 include a plurality of interchangeable pyramidal magnets. In one embodiment, the second plurality of magnets 106 are permanent magnets. In one embodiment, the second plurality of magnets 106 are configured to move towards and away from the first plurality of magnets (102 and 104) for varying the magnetic field provided by the first plurality of magnets (102 and 104) to the patient on present on the surgical bed.

In one embodiment, the system 100 further comprises one or more ultrasound probes 116 positioned between the first plurality of magnets (102 and 104) to measure the depth of a thoracic spinal cord of the patient 118. In one embodiment, the second plurality of magnets 106 are coupled to one or more reciprocating units 108 and provided operatively below the surgical bed 110. In one embodiment, the reciprocating units 108 are configured for facilitating the reciprocal movement of the second plurality of magnets 106. In one embodiment, the reciprocal movement of the second plurality of magnets 106 directs the magnetic field of the first plurality of magnets (102 and 104) substantially perpendicular to the surface 111 of the surgical bed 110 for entering into the patient's body 118 present on the surface 111 of the surgical bed 110. In one embodiment, the second plurality of magnets 106 are moved towards and away from the patient's body 118 for facilitating the variation of the magnetic field provided to the patient 118. The change in the magnetic field 128 induces an electric flux within the spinal cord 120 and/or spinal nerve roots that disturbs the neurons in the spinal cord 120, thereby preventing the patient 118 from feeling any pain during a surgical procedure.

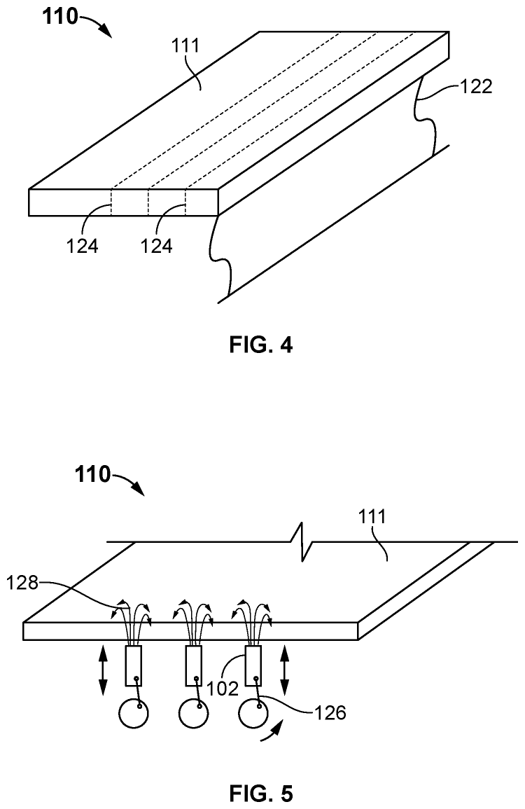

Referring to FIGS. 4-5, an alignment of one or more permanent magnets 102 under the surgical bed 110 is illustrated. Each permanent magnet 102 is connected to a piston 126 along the long axis of the surgical bed 110, and within 3 planes, that is, sagittally aligned—para-median 124. The surgical bed 110 has a base 122. The permanent magnets 102, ultrasound probe 116, and the second plurality of magnets 106 are positioned inside the base 122. In one embodiment, the permanent magnets 102 are connected to pistons 126 or other comparable devices configured to facilitate an off-axis rotational movement or wobbling movement (back and forth movement relative to the long axis of the surgical bed 110) thereof. In one embodiment, the pistons 126 bring the permanent magnets 102 within millimeters of the surface 111 of the surgical bed 110 before retracting the permanent magnets 102 away from the surface 111 of the surgical bed 110, that is, the underside of the surgical bed 110. In an operational configuration, the permanent magnets 102 reciprocate very quickly, for example, similar to the pistons of an internal combustion engine. Thus, as the permanent magnets 102 approach the surface 111, its magnetic field 128 spreads out perpendicular to the surface 111, and enters the body of the patient 118 present on the surgical bed 110.

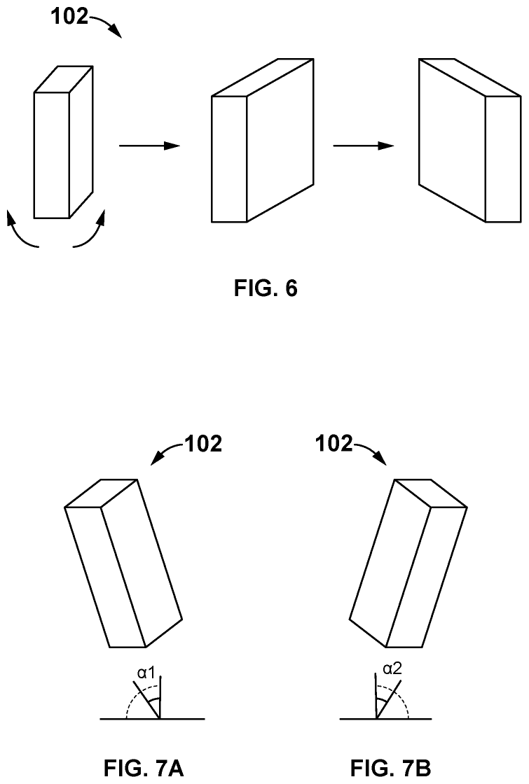

Referring to FIGS. 6 and 7A-7B, rotation of the permanent magnets 102 and angle of rotation of the permanent magnets 102 are illustrated. The permanent magnets 102 are aligned in the para-median axis 124, just off the center line. In some embodiments, the permanent magnets 102 may be arranged as three rows of permanent magnets 102. The permanent magnets 102 are aligned along the center line of the long axis of the surgical bed 110. In one embodiment, at least two “columns” of moving magnets is the minimum number of “columns” of permanent magnets 102 that may be needed to effectuate the varying magnetic field 128 into the body of the patient 118, specifically, the spinal cord 120 and the ventral and dorsal roots of the spinal nerves. A column of permanent magnets herein refers to multiple permanent magnets distributed along the length of the surgical bed in a spaced apart configuration.

In one embodiment, the permanent magnets 102 may be elongated rectangular-shaped bar magnets that may be arranged along the para-median axis 124. In one embodiment, the permanent magnets 102 may rotate by themselves for a few degrees off their long axis, so that the varying flow of magnetic field 128 is directed either perfectly perpendicular to the surface 111 of the surgical bed 110 or angled a few degrees cephalad (toward the head or anterior end of the body) or caudad (toward the tail or posterior end of the body) and/or lateral or medial. In one embodiment, the permanent magnets 102 wobbles (back and forth movement) relative to the long axis of the surgical bed 110. Thus, the varying magnetic field 128 can be at any one time or another, within a parameter of influence, similar to the “clouds of patterns” in which electrons move.

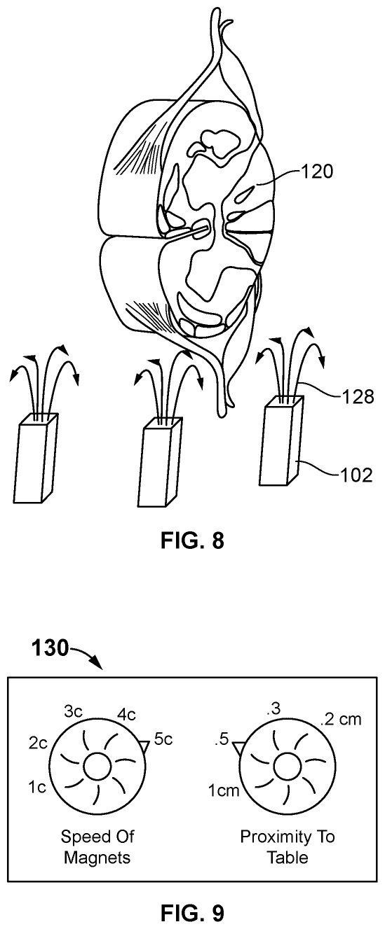

Referring to FIG. 8, magnets in relation to a cross section of the spinal cord are shown to emphasize the direction of the magnetic flux in relation to the spinal cord. In exemplary embodiments, an alignment of a plurality of columns of permanent magnets 102 may be placed along a length of a surgical bed 110 so that the magnets are positioned along a length of a patient's spine. The alignment of three columns of permanent magnets 102 along the long axis of the surgical bed 110 is configured to emanate the subsequent magnetic field from the permanent magnets 102. In one embodiment, the magnetic field 128 is directed towards the central spinal cord tissue and also along the ventral and dorsal root nerves, which ultimately carry the nerve impulses from the body periphery to the spinal cord 120, and the impulses from the spinal cord 120 to the periphery.

In one embodiment, the permanent magnets 102 are powerful rectangular elongated magnets and also of Halbach design. The magnetic field 128 is emanated from both types of magnets in a predictable and focused manner. The magnetic field 128 emanating outwards has the ability to create an electric field flux within a circuit found within the spinal cord 120, and/or the spinal cord 120 and spinal nerve roots.

In one embodiment, the first plurality of magnets (102 and 104) may provide the magnetic field to the patient 118 in an area corresponding to a cervical region all the way down to the beginning of Corda equine, i.e., actual spinal cord ends around lower thoracic/upper lumbar region.

Referring to FIG. 9, a first control panel 130 for controlling one or more parameters of the first plurality of magnets (102 and 104) and the second plurality of magnets 106 is illustrated. In one embodiment, the parameters include a speed at which the first and second plurality of magnets (102, 104, and 106) reciprocate near the surface 111 of the surgical bed 110.

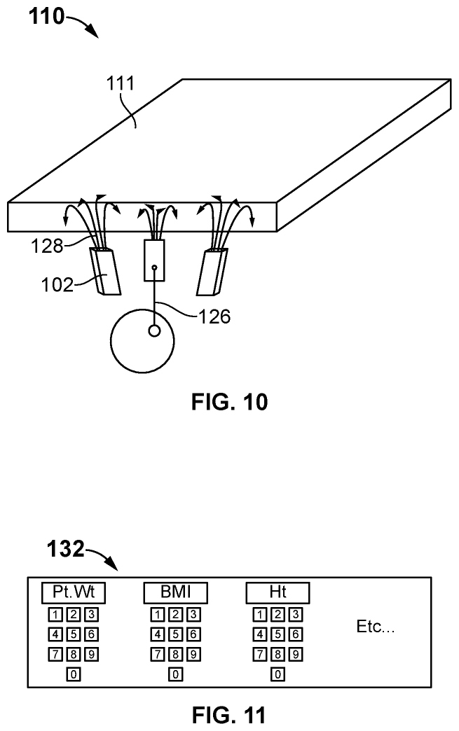

Referring to FIG. 10, a direction of the magnetic field 128 towards the surgical bed 110 is illustrated. The magnetic field influence can be changed in a certain range. In one embodiment, the desired range of spinal cord 120 that the operator has to influence is lower cervical region through upper thoracic region, or upper thoracic region through upper lumbar region, etc. In one embodiment, the direction of the magnetic field 128 is directly perpendicular to the surgical bed 110 or “off-center” within the “cloud range” of target area of the patient 118. For this, the permanent magnets 102 are oriented slightly off the center axis.

Referring to FIG. 11, a second control panel 132 configured to collect one or more parameters of the patient 118 is illustrated. In one embodiment, the parameters may be the patient's BMI, height, weight, and girth (side to side width). Once these parameters are set using the second control panel 132, the maximum magnetic field 128 and thus the maximum electric field flux thrust upon the spinal cord/nerves cannot be changed without manual adjustment by the operator. With most body sizes, the average depth of the spinal cord 120 from the skin may be within certain consistent ranges. However, individual patients vary, and, if the desired effects are not achieved with the “pre-set settings”, then certain variables such as the frequency of the permanent magnets 102 going up and down may need adjustments. Thus, the frequency of the magnetic field 128 affecting the target area of the patient 118 can be varied, and a distance of travel of the first plurality of magnets (102 and 104) towards the surface 111 of the surgical bed 110 can also be varied. Further, angle of the magnetic field from the first plurality of magnets (102 and 104) relative to the surface 111 of the surgical bed 110 can be manipulated. In one embodiment, a certain number of magnetic field 128 (the frequency of the up and down movement of the magnets) is required to affect the inter-spinal circuits as well as to maintain the effect of the electric field. In one embodiment, the electric field is induced from the magnetic field 128 in order to disrupt the sensory function of the spinal nerve roots and spinal cord 120.

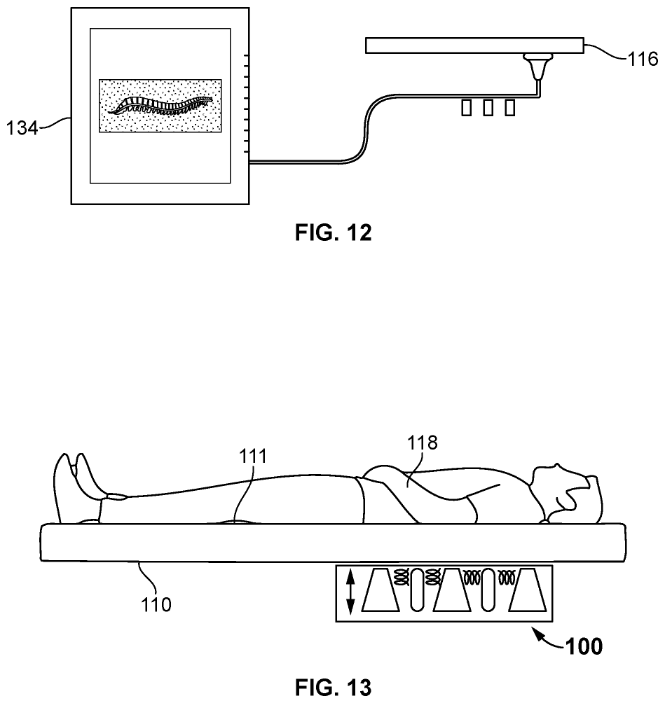

Referring to FIG. 12, an ultrasound image 134 of the spinal cord 120 is illustrated. After the patient's parameters are entered into the second control panel 132, the ultrasound probe 116 is moved into position. The permanent magnets 102 are retracted within the base 122 when the ultrasound probe 116 is in use. The base 122 is the area closest to the floor. In one embodiment, the ultrasound probe 116 may “sound” the patient's body and determine the depth of a patient's spinal cord 120. In one embodiment, the ultrasound probe is 116 is moved in a position configured to transmit a high-frequency sound waves around a target area of the patient's body for determining the depth of the patient's spinal cord 120. In one embodiment, the depth of spinal cord 120 is compared to the pre-programmed indices that are provided in a memory of the second control panel 132. Such body measurement corresponds to a depth of spinal nerve roots in the spinal cord. Once a quick ultrasound measurement is made, the ultrasound probe 116 is retracted and the permanent magnets 102 are then usable for providing the varying magnetic field to the patient 118 to affect the target tissue of the patient 118 such as the spinal cord and spinal nerve roots.

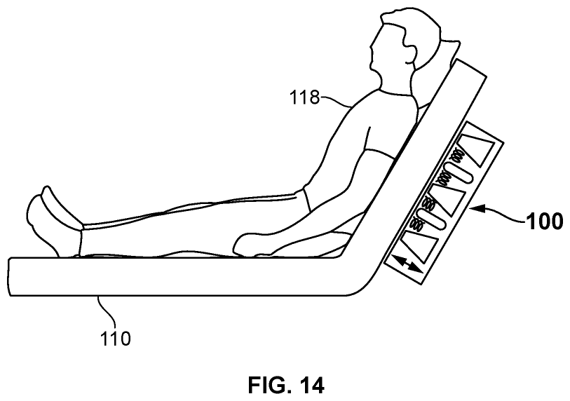

Referring to FIGS. 13-14, the system 100 arranged under various types of surgical beds 110 are illustrated. In one embodiment, the system 100 further comprises a cooling mechanism. The cooling mechanism is utilized for cooling the first and second plurality of magnets (102, 104, and 106) in order to amplify the power of the permanent magnets 102. In one embodiment, the repeated and rapid reciprocating movement of the second plurality of magnets 106 produces heat. The cooling mechanism is configured to reduce the heat generated due to the action of repeated and rapid reciprocating movement of the second plurality of magnets 106. The cooling mechanism is used to cool the first and second plurality of magnets (102, 104, and 106) to keep them functioning properly. In one embodiment, the cooling mechanism is configured to work when the surgical bed 110 is flat, that is, the flat surface 111 of the surgical bed 110 is parallel to the ground (as shown in FIG. 13) or if the surgical bed 110 is in a semi-Fowler's position (as shown in FIG. 14).

Advantageously, the system of the present invention comprises both electromagnets and permanent magnets that are positioned so that their combined magnetic field is applied along the spine of the patient. The system is configured to provide a change in the applied combined magnetic field that disrupts the neurons in the spinal cord. The disruption of the neurons prevents the patient from feeling pain/pressure from the surgical procedure.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A system for applying a magnetic field to anesthetize a patient, comprising:
a first plurality of magnets positioned below a surgical bed of the patient to apply a magnetic field when the patient present on a surface of the surgical bed;
a second plurality of magnets coupled to one or more reciprocating units and provided operatively below the surgical bed, the one or more reciprocating units configured to facilitate a reciprocal movement of the second plurality of magnets, thereby moving the second plurality of magnets towards and away from the patient's body for facilitating variation of the magnetic field provided to the patient; and
one or more ultrasound probes positioned between the first plurality of magnets to measure the depth of a thoracic spinal cord of the patient;
wherein the variation of the magnetic field induces an electric flux within the spinal cord and spinal nerve roots, thereby disrupting neurons in the spinal cord and preventing the patient from feeling any pain during a surgical procedure.

2. The system of claim 1, wherein the first plurality of magnets includes one or more permanent magnets and one or more electromagnets.

3. The system of claim 2, wherein the one or more permanent magnets are connected to one or more pistons to facilitate an off-axis rotational movement thereof, wherein the reciprocal movement of the second plurality of magnets directs the magnetic field of the first plurality of magnets substantially perpendicular to a surface of the surgical bed for entering into the patient's body part present in the surface of the surgical bed.

4. The system of claim 2, wherein at least one electromagnet is placed at a first end of the surgical bed, and at least one electromagnet is placed at a second end of the surgical bed.

5. The system of claim 4, wherein both the electromagnets include a plurality of on and off frequencies configured to impart a pulse wave pattern of magnetic field at both a cervical and a lower thoracic region of the patient present on the surgical bed.

6. The system of claim 2, wherein the magnetic field emanating from the first plurality of magnets is directed towards a central spinal cord tissue and along ventral and dorsal root nerves.

7. The system of claim 1, wherein the magnetic field is directed to a desired range of spinal cord including the lower cervical region through the upper thoracic region.

8. The system of claim 1, wherein the second plurality of magnets includes interchangeable pyramidal magnets.

9. The system of claim 1, further comprises a first control panel configured to control one or more parameters of the first and second plurality of magnets.

10. The system of claim 1, further comprises a second control panel configured to collect one or more parameters of the patient.

11. The system of claim 1, wherein the ultrasound probe is moved in a position configured to transmit a high-frequency sound waves around a target area of the patient's body for determining the depth of the patient's spinal cord.

12. The system of claim 11, wherein the determined depth of the patient's spinal cord is compared to the pre-programmed indices provided in a memory of the second control panel.

13. The system of claim 1, further comprises a cooling mechanism for cooling the first and second plurality of magnets.

14. The system of claim 13, wherein the cooling mechanism is configured to reduce the heat generated due to the action of repeated and rapid reciprocating movement of the second plurality of magnets.
